(19) United States
(12) Patent Application Publication
(10) Pub. No.: US 2019/0302124 A1
(43) Pub. Date: Oct. 3, 2019

(54) Compositions and Methods for Identifying Subjects Who May Benefit From Treatment With Therapeutic Agents

(71) Applicant: Laboratory Corporation of America Holdings, Burlington, NC (US)

(72) Inventor: Jeffrey Shuster, Chapel Hill, NC (US)

(21) Appl. No.: 16/366,694

(22) Filed: Mar. 27, 2019

Related U.S. Application Data
(60) Provisional application No. 62/648,718, filed on Mar. 27, 2018.
(60) Provisional application No. 62/657,288, filed on Apr. 13, 2018.

(51) Int. Cl. G01N 33/68; C07K 16/42; C07K 16/46; C07K 16/32

(52) U.S. Cl. CPC G01N 33/6854; C07K 16/42; C07K 16/46; C07K 16/32; C07K 2317/31; C07K 2317/21; C07K 2317/24; C07K 2317/52; C07K 2317/56; C07K 2317/55

(57) ABSTRACT

Disclosed herein are compositions and methods for the identification of subjects who are likely to benefit from use of a therapeutic agent. The present invention utilizes the specificity of therapeutic monoclonal antibodies for a single epitope to determine whether a therapeutic monoclonal antibody interacts with biological material in/from a patient.

### CROSS REFERENCE TO RELATED APPLICATION

The present application claims priority to U.S. Provisional Application No. 62/648,718, filed on Mar. 27, 2018 and U.S. Provisional Application No. 62/657,288, filed on Apr. 13, 2018, each of which are hereby incorporated by their entireties herein.

### FIELD OF THE INVENTION

This invention relates to compositions and methods for the identification of subjects who may benefit from treatment with therapeutic monoclonal antibodies.

### BACKGROUND

During drug development and in later patient care, it is useful to test clinical samples for the presence and or level of expression of the target protein for which a monoclonal antibody (mAb) has been designed to interact. Although mAbs are raised to specific analytes (usually proteins or portions of proteins), each mAb may react with a different epitope of the target analyte. It may also be the case that different mAbs may react with the same epitope, but by the nature of the creation of the mAbs, the mAbs could have different sequences and different affinities to the analyte of interest.

Since each mAb represents a unique material, the most efficient manner to address if a specific therapeutic mAb would interact with a biological material in/from a patient would be to create an assay comprising the same therapeutic mAb analyte binding region as a preferred assay reagent. If another assay reagent type is used, for example another antibody reagent, there is a high probability that this second assay reagent will be to a different analyte epitope and will therefore not be as specific to identify the expected interactions between the patient material (in vivo or in vitro) and the therapeutic mAb.

In current practice, companies that develop mAb pharmaceuticals often co-investigate and/or co-develop immunohistochemistry (IHC) tests based on antibodies that have been made to the same protein as that targeted by the mAb pharmaceutical. This is often done by a search of current vendor catalogs for reagents (e.g., monoclonal or polyclonal antibodies). If one(s) is found, the company will test the catalog product to assess the characteristics of the product in IHC. In addition to the technical issues described above (different epitope), the commercial supply may not be suitable for use in development of a clinical test due to any number of factors including unstable source material (such as an unstable hybridoma), non-quality manufacturing, intellectual property right matters, and/or commercialization rights. For these reasons, current screening and identification techniques are prone to producing both false positives and false negatives. If no suitable reagents can be found, the company may choose to create a new Ab reagent by standard methods in the art. This will have many of the same technical disadvantages, and in addition can take 6-12 months to develop, thereby slowing a drug development project. Therefore, methods of identifying which patients are most likely to benefit from therapeutic agents are needed.

### BRIEF SUMMARY OF THE INVENTION

Embodiments of the invention comprise compositions and methods for the identification of subjects who may benefit from a therapeutic agent. The invention may be embodied in a variety of ways.

In some aspects, the invention comprises a composition of a targeting complex for the identification of subjects who may benefit from a therapeutic agent. In some embodiments, the targeting complex comprises antibody A and antibody B. Antibody A further comprises a potential therapeutic monoclonal antibody or fragment thereof. In certain instances the monoclonal antibody may comprises a variable region specific to an epitope of interest and a human constant region. Antibody B further comprises an antibody that recognizes the human constant region of antibody A. Antibody A and antibody B may be bound together to form a targeting complex.

In another aspect, the invention comprises methods for making a targeting complex. Antibody A may be reacted with Antibody B to produce a targeting complex. Once antibody A is complexed to antibody, B the targeting complex if purified to remove any unbound reagents.

In another aspect, the invention comprises methods for identifying subjects who may benefit from treatment with a therapeutic agent. The therapeutic agent may comprise a monoclonal antibody or a portion thereof. Steps for identifying subjects who may benefit from treatment with a therapeutic agent comprise: (i) obtaining a sample, (ii) incubating the sample with a targeting complex, (iii) detecting the antigen-antibody complex; and (iv) predicting whether the subject may be responsive to the therapeutic agent. The targeting complex may comprise antibody A and antibody B. Antibody A further comprises a potential therapeutic monoclonal antibody or fragment thereof. In certain instances the monoclonal antibody may comprises a variable region specific to an epitope of interest and a human constant region. Antibody B further comprises an antibody that recognizes the human constant region of antibody A.

### BRIEF DESCRIPTION OF THE DRAWINGS

FIG. 1 shows a preferred embodiment for the detection of expression of a tumor protein via the target epitope (epitope A) of the therapeutic monoclonal antibody (AbA).

FIG. 2 shows the interaction between a therapeutic monoclonal antibody (AbA) and the specific epitopes (epitope A) of the tumor proteins.

FIG. 3 shows the detection of expression of a tumor protein via an epitope (epitope C) that is not the target of the therapeutic monoclonal antibody.

FIG. 4 shows a preferred embodiment of an antigen-binding assay for the identification of subjects who may benefit from a therapeutic monoclonal antibody.

FIG. 5 shows a preferred embodiment of an antigen-binding assay for the identification of subjects who may benefit from a plurality of differing therapeutic monoclonal antibodies.

### DETAILED DESCRIPTION OF THE INVENTION

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. Known methods and techniques are generally performed according to conventional methods well-known in the art and as described in various general and more specific references that are discussed throughout the present specification unless otherwise indicated. Enzymatic reactions and purification techniques are performed according to manufacturer's specifications, as commonly accomplished in the art or as described herein. The nomenclatures used in connection with the laboratory procedures and techniques described herein are those well-known and commonly used in the art.

The following terms, unless otherwise indicated, shall be understood to have the following meanings:

As used herein, the terms “a”, “an”, and “the” can refer to one or more unless specifically noted otherwise.

The use of the term “or” is used to mean “and/or” unless explicitly indicated to refer to alternatives only or the alternatives are mutually exclusive, although the disclosure supports a definition that refers to only alternatives and “and/or.” As used herein “another” can mean at least a second or more.

Throughout this application, the term “about” is used to indicate that a value includes the inherent variation of error for the device, the method being employed to determine the value, or the variation that exists among samples.

The term “biological sample” refers to a sample of tissue or fluid isolated from a subject including, but not limited to, for example, blood, plasma, serum, fecal matter, urine, bone marrow, bile, spinal fluid, lymph fluid, samples of the skin, external secretions from the body, such as from skin, respiratory, intestinal and genitourinary tracts, tears, saliva, milk, blood cells, organs, biopsies and also samples in vitro cell culture constituents, for example conditioned media resulting from the growth of cells and tissues in culture medium, for example recombinant cells and cell components.

The term “indicator” or “indicator moiety” or “detectable moiety” or “detectable biomolecule” or “reporter” or “label” refers to a molecule that provides a signal that can be measured in a qualitative or quantitative assay. For example, an indicator moiety may comprise an enzyme that may be used to convert a substrate to a product that can be measured. An indicator moiety may be an enzyme that catalyzes a reaction that generates bioluminescent emissions (e.g., luciferase, HRP, or AP). Or, an indicator moiety may be a radioisotope that can be quantified. Or, an indicator moiety may be a fluorophore. Or, other detectable molecules may be used.

The term “antigen” refers to a molecule or a set of molecules on a cell surface, or a complex that includes such molecule or set of molecules, with which the antigen combining site of a monoclonal antibody useful for the present purposes binds.

The terms “polypeptide”, “peptide”, or “protein” refer to designate a linear series of amino acid residues connected one to the other by peptide bonds between the alpha-amino and carboxy groups of adjacent residues.

The term “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The terms “therapeutic agent” or “potential therapeutic agent” refer to a substance capable of producing a curative effect in a disease state.

The present invention utilizes the specificity of therapeutic monoclonal antibodies for a single epitope to identify subjects who may benefit from the same therapeutic monoclonal antibody. Antibodies can be used for identification at the protein level; however, each protein will have a variety of epitopes. A particular monoclonal antibody may only be specific for a single variety of epitope. Companies offer a large number of antibodies against proteins, but the specificity of these antibodies varies greatly. Antibodies targeting different parts (epitopes) of a protein can have opposing effects. Identification of individuals who overexpress the target protein of a particular therapeutic can be helpful for identifying suitable candidates for treatment with the therapeutic. However, in certain instances, an individual who overexpresses the target protein may not be a suitable candidate for treatment with a particular therapeutic if they do not express the specific epitope of the protein targeted by the therapeutic.

Each of the embodiments of the compositions, methods, and systems of the invention can be used to determine whether a specific therapeutic agent would interact with a biological material in/from a patient. Methods according to the present invention can be performed in a shortened time with increased specificity and concordance.

### I. Targeting Complex

As described herein, the compositions, methods, and systems of the invention may comprise a targeting complex for use in identifying subjects who may benefit from therapeutic agents. In some embodiments, the targeting complex comprises two antibodies: Antibody A and Antibody B. Antibody A further comprises a potential therapeutic monoclonal antibody or fragment thereof. In certain instances the monoclonal antibody may comprises a variable region specific to an epitope of interest and a human constant region. Antibody B further comprises an antibody that recognizes the human constant region of antibody A.

There are a number of different antibody isotypes, the most common being immunoglobulin G (IgG). IgG antibodies are composed of two polypeptide chains: a heavy chain and a light chain. Each heavy chain has two regions: the constant region and the variable region. All antibodies of the same isotype are composed of the same constant region. The fragment-antigen binding (Fab) is composed of one constant and one variable domain of each of the heavy and the light chain and can be separated from the fragment crystallizable (Fc) portion of the molecule. The Fc portion of the molecule acts as a binding site for receptors on the surface of lymphocytes and secondary antibodies. Fab fragments contain variable domains, which consist of antibody hypervariable domains. Hypervariable domains determine the specificity of the antibody and binding capacity. The Fc region consists entirely of constant domains.

Both polyclonal and monoclonal antibodies can be produced using animals. When mammals are immunized with a particular immunogen (antigen), polyclonal antibodies are produced. Antibody-generating molecules (antigens) are protein molecules that induce an immune response (generation of antibodies). Polyclonal antibodies comprise a number of different IgGs, each capable of recognizing different epitopes on the same immunogenic protein. These IgG proteins can be isolated form the blood of immunized animals. Polyclonal antibodies can be raised in a variety of animals including, but not limited to mice, rabbits, rats, guinea pigs, donkeys, goats, or sheep. In some embodiments, it is preferable to use antibodies raised in mice or rabbits.

Similarly to the production of polyclonal antibodies, mAbs are produced by immunizing animals with a particular antigen. For the isolation of monoclonal antibodies, spleens are removed and the lymphocytes producing antibodies are isolated from the spleen. Each lymphocyte, producing a unique antibody, can be immortalized by fusion with tumor cells to produce clonal lines (hybridomas) capable of making each specific antibody. Each hybridoma is capable of producing monoclonal antibodies specific for only a single immunogenic epitope. MAbs are raised against a specific antigen of interest (e.g., an antigen found on the surface of tumors). Tumor cells displaying proteins with unique epitopes can be targeted by monoclonal antibodies specific for the epitope. The immune system makes antibodies against specific epitopes on immunogens, but not against the entire immunogen (Ag). Distinct antibodies can be generated that recognize the same protein, but different epitopes. MAbs are identical such that they bind to only one substance (e.g., a specific epitope). An epitope is the specific region of an antigen to which antibodies bind. In determining whether a therapeutic mAb is likely to be effective, it is important to determine whether the specific therapeutic mAb would interact with the biological material in/from a patient. In some instances therapeutic monoclonal antibody (AbA) is able to interact with a specific type of epitope (epitope A). However, AbA is specific only for epitope A and not other epitopes on the protein (e.g., epitope C). To identify patients who are likely to benefit from therapeutic AbA, it is important to identify subjects who have biologic material (epitope A) that is reactive with the therapeutic.

In some embodiments, antibody A is a mAb or a fragment thereof. In certain embodiments, the fragment is Fab. In some instances, the monoclonal antibody, or fragment thereof, can have a therapeutic effect. Therapeutic monoclonal antibodies can be murine, humanized, chimeric, or fully human. In a preferred embodiment, the mAb comprises a constant region of human sequence. The constant region of an antibody determined the mechanism used to eliminate the antigen.

In some embodiments, the therapeutic mAb comprising a human constant region is a chimeric antibody. Chimeric mAbs are composed of non-human animal variable regions fused onto a human constant region. This fusion allows chimeric mAbs to retain the specificity and affinity for the specific antigen. Chimeric mAbs possess a fully human constant region and therefore exhibit decreased immunogenic effect in humans. In some embodiments, the variable regions of the chimeric mAbs are produced in mammals including, but not limited mice, rabbits, rats, guinea pigs, donkeys, goats, or sheep. In a preferred embodiment, the variable regions are mouse.

In other embodiments, the therapeutic monoclonal antibody comprising a human constant is a humanized mAb. In some instances, therapeutic mAbs may be humanized in order to decrease the non-human portion of antibodies. MAbs are humanized by replacing the hypervariable loops of a human antibody with the hypervariable loops of a non-human animal antibody. Similarly, to chimeric mAbs, the non-human animal portion of the mAb is responsible for binding the target antigen. Unlike chimeric antibodies, only the hypervariable regions, responsible for binding to epitopes, are non-human, thereby, further decreasing the immunogenic effect in humans. In some embodiments, the hypervariable regions of the humanized mAbs are produced in mammals including, but not limited mice, rabbits, rats, guinea pigs, donkeys, goats, or sheep. In a preferred embodiment, the hypervariable regions are mouse.

In other embodiments, the therapeutic monoclonal antibody comprising a human constant region is fully human. Transgenic mice or phage display libraries can be used to produce fully human monoclonal antibodies. Segments of human Ig genes can be introduced into the genome of mice lacking mouse antibody production. When the transgenic mice are introduced to a specific immunogen, the mice produce significant amounts of fully human antibodies. Fully human antibodies are advantageous in that they contain no non-human portions and, therefore, do not create an immunogenic effect when used as therapeutics in human subjects.

In some embodiments of the invention, the monoclonal antibody has been approved by the FDA for use as a therapeutic. In other aspects of the invention, the monoclonal antibody is one that is undergoing testing for use as a therapeutic or has potential for use as a therapeutic. A number of monoclonal antibodies have been approved by the FDA for therapeutic use including, but not limited to abciximab, adalimumab, adotrastuzumab emtansine, alemtuzumab, alirocumab, atezolizumab, avelumab, basiliximab, belimumab, bevacizumab, bezlotoxumab, blinatumomab, brentuximab vedotin, broadalumab, canakinumab, capromab pendetide, certolizumab pegol, cetuximab, daclizumab, daratumumab, densosumab, dinutuximab, durvalumab, elotuzumab, evolocumab, golimumab, infliximab, ipilimumab, ixekizumab, mepolizumab, natalizumab, necitumumab, nivolumab, obinutuzumab, ocrelizumab, ofatumumab, olaratumab, pertuzumab, ramucirumab, rituximab, siltuximab, tocilizumab, trastuzumab, ustekinumab, vedolizumab, sarilumab, and benralizumab. To date, FDA-approved therapeutic mAbs contain variable regions that are mouse, rat, or human.

In some embodiments, the targeting complex further comprises a second antibody: antibody B. Antibody B recognizes the human constant region of antibody A. Antibody B is a monoclonal or polyclonal antibody raised against the human region of antibody A. In a preferred embodiment, antibody B is a monoclonal antibody. Antibody B can be raised in mammals including, but not limited mice, rabbits, rats, guinea pigs, donkeys, goats, or sheep. In a preferred embodiment, antibody B is raised in mice or rabbits.

In some embodiments, Antibody B is a secondary antibody specific for antibody A. Secondary antibodies may be polyclonal or monoclonal and may be specific for whole Ig molecules, or fragments thereof, such as the Fc or Fab regions. In certain instances, the secondary antibody is raised against the human constant region of the targeting agent. Secondary antibodies are raised against the host species used to generate the primary antibody. For example, primary antibodies raised in mouse require secondary anti-mouse secondary antibodies raised in a host species other than mouse (e.g., rabbit anti-mouse secondary antibodies). Detection of secondary antibodies can be accomplished in a variety of ways. Most commonly, secondary antibodies may be conjugated to an enzyme or fluorescent proteins or dyes. Therefore, in some embodiments, the targeting complex comprises a detection moiety.

In alternative embodiments, the targeting complex does not comprise a detection moiety. In these cases, the targeting complex functions as a primary antibody, which may be detected at a later stage with a third antibody, antibody C.

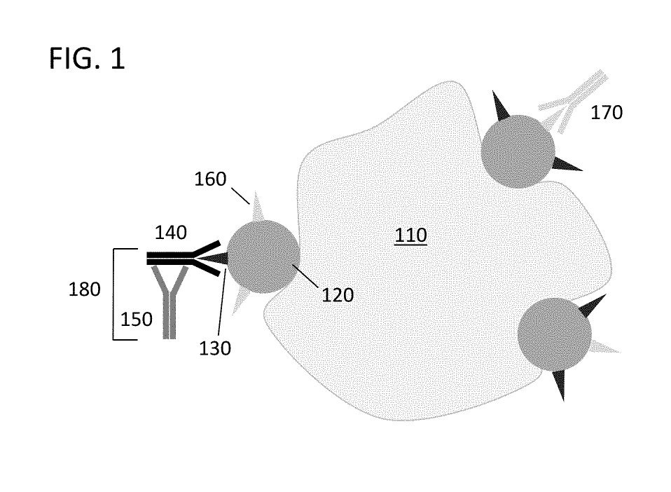

FIG. 1 shows the targeting complex 180 comprising antibody A (AbA) 140 bound to antibody B (AbB) 150. Antibody B 150 recognizes the human constant region of antibody A 140. Through antibody A, the targeting complex is able to bind to one specific type of epitope (epitope A) 130 of a tumor surface protein 120 on a tumor 110. However, the targeting complex is not able to bind to any other type epitope located on the surface protein (e.g., epitope C) 160. Other monoclonal antibodies that are specific for the same protein as AbA, but a different epitope, are able to bind other types of epitopes. For example, FIG. 1 shows AbC 170 bound to the same surface protein 120, but a different type of epitope (epitope C) 160.

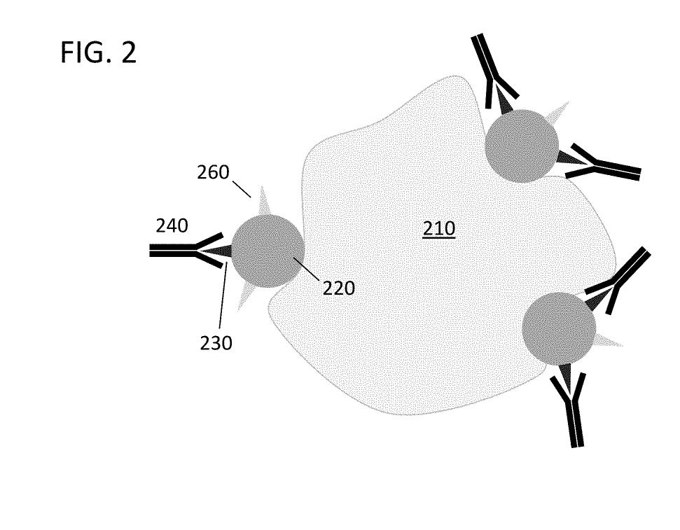

FIG. 2 shows therapeutic monoclonal antibody (AbA) 240 is able to interact with a specific type of epitope (epitope A) 230. However, AbA 240 is specific only for epitope A 230 and not other epitopes on the protein (e.g., epitope C) 260. To identify patients who are likely to benefit from therapeutic AbA 240, it is important to identify subjects who have biologic material (epitope A) 230 that is reactive with the therapeutic.

In certain embodiments, the secondary antibody may be conjugated with a detection moiety. The choice of label depends upon the application and the desired method of detecting the antibody. In some embodiments, the method of detection may be Western blot, ELISA, immunohistochemistry, immunocytochemistry, immunofluorescence, or flow cytometry. In some embodiments, the detection moiety may generate light and/or may be detectable by a color change. Detection signals may be generated using an enzyme such as alkaline phosphatase (AP) or horseradish peroxidase (HRP), or a fluorescent molecule (fluorophore), or a bioluminescent molecule (NANOLUC®). In some embodiments positive detection can be indicated by a change in color or fluorescence. Fluorescent proteins naturally fluoresce (intrinsic fluorescence or autofluorescence) by emitting energy as a photon when the fluorescent moiety containing electrons absorb a photon.

Various appropriate enzymes are commercially available for use as a detection moiety, such as alkaline phosphatase (AP), horseradish peroxidase (HRP), or luciferase (Luc). The indicator moiety may react with a substrate to emit a detectable signal. One or more signal producing components can be reacted with the indicatory moiety to generate a detectable signal. If the indicator moiety is an enzyme, then detection is obtained by reacting the enzyme with one or more substrates or additional enzymes and substrates to produce a detectable reaction product. In an alternative signal producing system, the label can be a fluorescent compound where no enzymatic manipulation of the label is required to produce the detectable signal. Fluorescent molecules including, for example, fluorescein and rhodamine and their derivatives and analogs are suitable for use as labels in such a system. In yet another alternative embodiment, the indicator moiety can be a cofactor, then amplification of the detectable signal is obtained by reacting the cofactor with the enzyme and one or more substrates or additional enzymes and substrates to produces a detectable reaction product.

For example, trastuzumab, is a humanized mAb from mouse. Trastuzumab is an FDA approved therapeutic monoclonal antibody for the treatment of HER2-positive breast cancer. Trastuzumab binds the HER2 protein, which is overexpressed by some breast cancer cells, thereby, blocking the receptor from receiving growth signals. Trastuzumab is reactive to a single HER2 epitope (Her2 extracellular domain IV juxtamembrane epitope). HER2 receptor proteins have a number of different epitopes to which trastuzumab does not interact. The constant regions of trastuzumab are human and the hypervariable regions responsible for binding HER2 are mouse. In one embodiment of the invention, a mouse anti-human mAb raised against the constant region of trastuzumab (detection antibody) may be reacted with trastuzumab (therapeutic agent) to produce a targeting complex for identifying subjects who are likely to benefit from treatment with trastuzumab. In some embodiments, the detection antibody maybe conjugated to a detection moiety as described above.

### II. Methods of Making Targeting Complex

Another aspect of the invention comprises methods for making a targeting complex capable of detecting a specific protein epitope. In some embodiments, the targeting complex is produced by reacting antibody A with antibody B. Once antibody A is complexed to antibody, B the targeting complex if purified to remove any unbound reagents.

Reacting Therapeutic Target with Detection Antibody

In some embodiments, a method for making a targeting complex comprises reacting antibody A with antibody B. Antibody A and antibody B. Antibody A further comprises a potential therapeutic monoclonal antibody or fragment thereof. In certain instances the monoclonal antibody may comprises a variable region specific to an epitope of interest and a human constant region. Antibody B further comprises an antibody that recognizes the human constant region of antibody A. In certain embodiments, antibody B is a monoclonal or polyclonal antibody.

In some embodiments, antibody B is a secondary antibody. Secondary antibodies are available in a variety of formats including, but not limited to liquid, lyophilized, and with additives (e.g., glycerol). Liquid secondary antibodies are generally concentrated and will require dilution according to the manufacturer's instructions. Dry lyophilized secondary antibodies may be reconstituted with diluents. Secondary antibodies (e.g., glycerol and BSA) may contain additives to stabilize and extend their shelf life. In some embodiments, a blocking agent is used as the diluent (e.g., 1% BSA in PBST).

In some embodiments, secondary antibodies may be conjugated to labels. In alternative embodiments, secondary antibodies may be in unconjugated forms.

Any method or technique commonly known or well-used in the art may be used to form a complex of antibody A and antibody B. Generally, antibody A is incubated with antibody B for at least 15, 20, 25, 30, 35, 40, 45 minutes, or longer. In some embodiments, Antibody A is incubated Antibody B at about room temperature (15-25° C.). In some embodiments Antibody and Antibody B are incubated overnight at about 4° C.

### Purification of the Targeting Complex

In some embodiments, the targeting complex is purified to remove all unbound antibody A and unbound antibody B using standard methods generally known in the art. For example, the targeting complex may be purified using antibody purification resins including, but not limited to Protein A, Protein G, Protein A/G, Protein L, or Melon Gel. In some embodiments, immunoprecipitation may be used for small-scale affinity purification. Alternatively, large scale process chromatography can be used to purify the targeting complex.

### III. Antigen-Binding Assay

An antigen-binding assay can be used to determine whether a subject may benefit from treatment with a therapeutic agent. An ideal assay for identifying subjects who may benefit from a therapeutic agent is able to identify patients with biologic material that is capable of interacting with a specific therapeutic agent. Current assays do not use the therapeutic mAbs to identify subjects, but rather, use a commercially available antibody capable of interacting with the protein of interest. This alternate mAb may; however, be specific for a different analyte epitope and will therefore not be [as] specific to identify the expected interactions between the patient material (in vivo or in vitro) and the therapeutic mAb. False negative and false positives are both characteristic of the current methods used to identify subjects for treatment with pharmaceutical mAbs. False negatives could result in subjects who may benefit from the use of a particular therapeutic being withheld from treatment. While false positives could result in treatment of subjects who are unlikely to receive any benefit. Therefore, it is important to develop an assay that is able to accurately identify subjects who are likely to respond to a therapeutic agent.

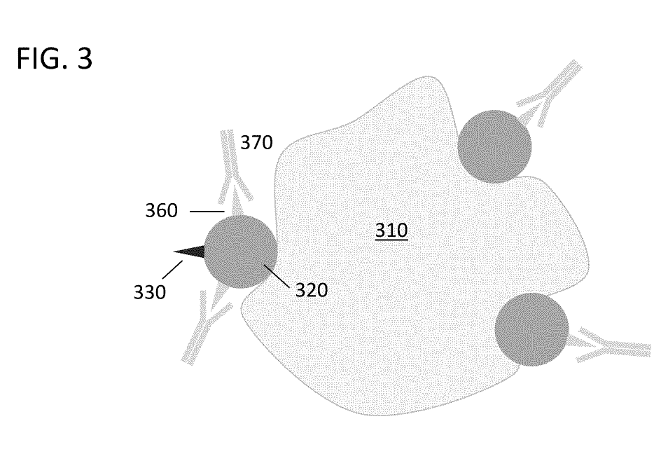

FIG. 3 shows a typical assay for identification of subjects who may benefit from a therapeutic agent. An antibody (AbC) 370, other than the therapeutic agent, that is specific for the same surface protein 320 as the therapeutic agent is used to determine expression of the surface protein 320 in the subject. However, these assays are not indicative of whether a biological sample in/from the subject will be reactive with the therapeutic agent because AbC 370 binds a different epitope (epitope C) 360.

For example, a number of diagnostics are used to identify subjects who are likely to benefit from treatment with trastuzumab. Some diagnostics for trastuzumab treatment use polyclonal antibodies specific for the HER2 protein. Tissue samples are incubated with the polyclonal antibody, and binding is visualized to determine the level of HER2 overexpression. However, unlike trastuzumab, these polyclonal antibodies are not specific for the unique epitope to which trastuzumab targets. The use of polyclonal antibodies is likely to be over-inclusive in identifying patients who are likely to benefit from a therapeutic. Polyclonal antibodies are able to bind to a variety of epitopes on a protein and are therefore more likely to identify a subject as overexpressing HER2 even if the biologic material of the subject is unlikely to interact with trastuzumab.

Alternatively, monoclonal antibodies specific for HER2 protein may be used to identify patients who overexpress HER2. However, these mAbs are specific for a HER2 epitope that differs from the epitope targeted by trastuzumab. Therefore, it is possible that a subject who is identified as overexpressing HER2 may lack the specific epitope with which trastuzumab interacts.

Methods disclosed herein relate to the selection or identification of subjects who may benefit from treatment with a specific therapeutic agent. Each of the embodiments of the compositions, methods, and systems of the invention allows for the identification and/or selection of an individual likely to benefit from a particular therapeutic agent. In some embodiments, the method for identifying subjects likely to benefit from treatment with a therapeutic agent comprises: (i) obtaining a sample; (ii) incubating the sample with a targeting complex to form an antigen-antibody complex; (iii) detecting the antigen-antibody complex; (iv) predicting subject responsiveness to the therapeutic agent. In some embodiments, the targeting complex comprises two antibodies: Antibody A and Antibody B. Antibody A further comprises a potential therapeutic monoclonal antibody or fragment thereof. In certain instances the monoclonal antibody may comprises a variable region specific to an epitope of interest and a human constant region. Antibody B further comprises an antibody that recognizes the human constant region of antibody A. In some embodiments, a plurality of differing targeting complexes may be used to predict subject responsiveness to multiple therapeutic agents with one assay. In some embodiments, the plurality of targeting complexes comprise AbA1, AbA2, AbA3, and so on. Each antibody A may comprise different potential therapeutic monoclonal antibody, or fragment thereof and may further comprise a unique human constant region. The plurality of targeting complexes may further comprise AbB1, AbB2, AbB3, and so on. Each different antibody B may recognize the human constant region of its corresponding antibody A. For example, AbB1 may bind to the human constant region of AbA1.

### Samples

MAbs can be used to treat a variety of diseases, most commonly, cancer or autoimmune disease. Subjects who are considered suitable for treatment are those subjects who are expected to benefit from or respond to the treatment. In some embodiments, samples are obtained from subjects who have, or are suspected of having, or are at risk of having cancer or autoimmune disease. The cancer may be breast cancer, B-cell chronic lymphocytic leukemia, urothelial carcinoma, non-small cell lung cancer, Merkel Cell carcinoma, Mantle Cell lymphoma, colorectal cancer, precursor B-cell acute lymphoblastic leukemia, Hodgkin's lymphoma, non-Hodgkin's lymphoma, prostate cancer, multiple myeloma, melanoma, Chronic Lymphocytic Leukemia, Acute Myeloid Leukemia, neuroblastoma, soft tissue sarcoma, gastric cancer, cervical cancer, renal cell carcinoma, or any other cancer for which a therapeutic agent is available. The autoimmune disease may be rheumatoid arthritis, juvenile idiopathic arthritis, psoriatic arthritis, Crohn's disease, ulcerative colitis, plaque psoriasis, systemic lupus erythematosus, or multiple sclerosis, or any other autoimmune disease for which a therapeutic agent is available. In other embodiments, samples are obtained from subjects who have any disease for which a monoclonal therapeutic antibody is available or being tested.

In some embodiments, the sample may comprise or be derived from blood, serum, tissue, biopsy, or cells isolated from an individual. In a preferred embodiment, the tissue sample may be taken from cancerous tumor tissue. Samples may be obtained by biopsy, scrapings, or surgical removal. Samples may be whole tissue sections. In certain embodiments, samples may be primary, metastatic, stage III, and stage IV disease specimens. In some embodiments, tumor tissue may be harvested via core biopsy or fine needle aspiration. Or, other samples as described herein may be used.

### Sample Preparation

Methods for fixing tissue samples are generally known in the art. The time required for fixation is dependent upon the size of the sample and the fixative used (e.g., neutral buffered formalin, glutaraldehyde, Bouin's or paraformaldehyde). In a preferred embodiment, the tissue sample is fixed with formalin. In some embodiments, the fixed sample is then embedded in paraffin to prepare a formalin-fixed and paraffin-embedded (FFPE) sample. In other embodiments, the tissue samples are embedded in other sectioning media. In some embodiments, the tissue samples are at least 1, 2, 3, or 4 mm thick. In a preferred embodiment, tissue samples are 3 mm thick.

In some embodiments, FFPE samples are sectioned, mounted, and dried on a microscope slide. Sectioned FFPE samples are at least 2, 3, 4, or 5 μm thick. In a preferred embodiment, sectioned samples are about 3 μm thick. Sectioned FFPE samples may be mounted and dried onto a slide. In a preferred embodiment, sectioned samples are mounted onto a Superfrost Plus® slide and dried at least 60, 65, 70, 75, 80, or 85° C. for less than 20, 19. 18, 17. 16, 15, 14, 13, 12, 11, 10, 9, 8, 7, 6, 5, 4, or 3 minutes. Or, other methods known in the art may be used.

### Detecting the Antigen-Antibody Complex

The methods for identifying subjects who may benefit from treatment with a therapeutic agent comprise detecting the antigen-antibody complex. In some embodiments, immunohistochemistry (IHC) can be used to detect the presence of the target epitope in the sample. The target epitope is the target of the therapeutic agent. Primary antibodies can be used to detect proteins within a tissue section. In some instances, proteins are detected directly using primary antibodies conjugated to detection moieties. In other instances, proteins are detected indirectly through conjugated secondary antibodies against the primary antibody.

The methods for identifying subjects who may benefit from treatment with a therapeutic agent comprise incubating the sample with a targeting complex to form an antigen-antibody complex. In some embodiments the targeting complex comprises: two antibodies: Antibody A and Antibody B. Antibody A further comprises a potential therapeutic monoclonal antibody or fragment thereof. In certain instances the monoclonal antibody may comprises a variable region specific to an epitope of interest and a human constant region. Antibody B further comprises an antibody that recognizes the human constant region of antibody A.

In some embodiments, antigen retrieval is conducted prior to incubation with the targeting complex. Fixation, while important for preservation of tissue morphology, can mask protein epitopes and prevent antibody interaction. Masking of an epitope can result from cross-linking within the epitope or peptides near the epitope. Antigen retrieval is a technique used to unmask an antigen to allow for epitope-antibody binding. Any method for antigen retrieval generally known in the art may be used. In some embodiments, antigen retrieval is heat-mediated. In other embodiments, antigen retrieval in enzymatic. The antigen retrieval process may require heating, pressure cooking, and/or protease treatment of samples in order to unmask antigens.

Following antigen retrieval, histological samples may be incubated with the targeting complex. In some embodiments the targeting complex is first diluted. The dilution of the targeting complex will depend on the therapeutic agent and the detection antibody. If the target of the therapeutic agent is present in the sample, the targeting complex will form an antigen-antibody complex.

For example, the therapeutic agent may be trastuzumab. In this instance, the targeting complex would comprise trastuzumab bound to an antibody that recognizes the human constant region of trastuzumab (antibody B), as described above. The trastuzumab targeting complex can be diluted as required to detect specific binding prior to incubation with histological samples for detection of HER2 protein.

Binding of the targeting complex to the target antigen may be determined directly or indirectly. In some embodiments, the targeting complex may comprise a detection antibody conjugated to an detection moiety, as described above. In some embodiments, the detection moiety comprises a fluorescent tag or an enzyme. The targeting complex, comprising antibody B conjugated to a detection moiety, binds to the antigen and the detection moiety can be visualized without further antibody interaction.

In other embodiments, antibody B is not conjugated to a detection moiety. Indirect IHC assays can be used to detect proteins. Indirect IHC assays involve unconjugated primary antibody binding to the antigen and then using labeled secondary antibody to bind to the primary antibody. Where the secondary antibody is conjugated to an enzymatic label, a chromagenic or fluorogenic substrate may be added to provide visualization of the antigen. In some embodiments, proteins are detected indirectly using secondary antibodies against the detection antibody of the targeting complex. The secondary antibody may be conjugated to a detection moiety. In some embodiments, each AbB may be conjugated to a different detection moiety. For example, AbB1 may be conjugated to a first detection moiety and AbB2 may be conjugated to a second detection moiety.

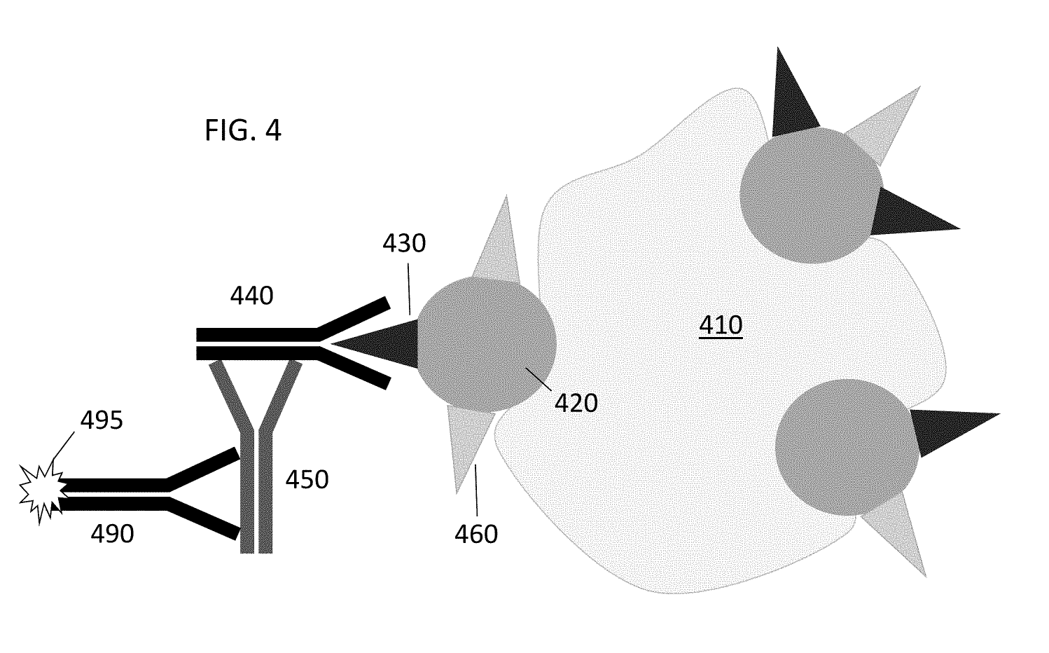

FIG. 4 shows detection of the target antigen (epitope A) 430 using a targeting complex comprised of antibody A (AbA) 440 and antibody B (AbB) 450. AbB 450 is not conjugated to a detection moiety. However, a secondary antibody (2° Ab) 490 can be conjugated to a detection moiety 495 and used to detect binding of the primary antibody 440 (targeting complex) to the antigen (epitope A) 430.

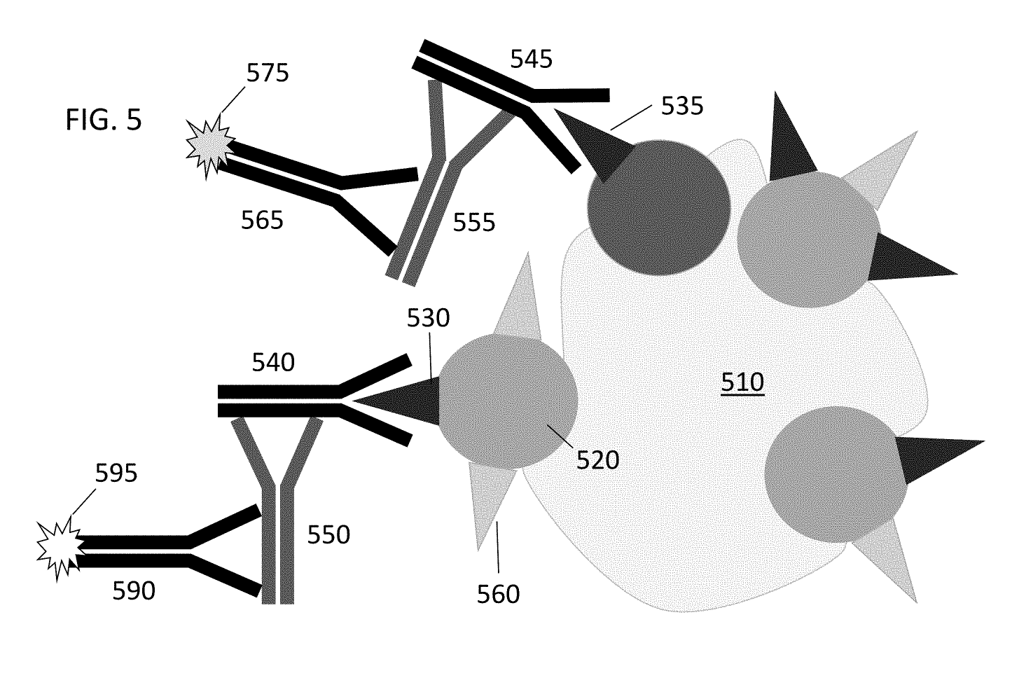

FIG. 5 shows detection of two different target antigens (epitope A1 530 and epitope A2 535) using a plurality of differing targeting complexes comprised of two different antibody As (AbA1 540 and AbA2 545) and two corresponding antibody Bs (AbB1 550 and AbB2 555). Two different secondary antibodies (2° Ab1 590 and 2° Ab2 565) can be conjugated to two corresponding detection moieties (1^{st} detection moiety 595 and 2^{nd} detection moiety 575) and used to detect binding of each primary antibody 540 and 545 (targeting complex) to each respective antigen (epitope A1 530 or A2 535).

A number of detection moieties are generally known in the art and are available to be used in either direct or indirect IHC assays. Detection moieties include, but are not limited to radioisotopes, fluorescent or chemiluminescent labels, and enzyme-substrate labels. Examples of enzyme-substrate labels include luciferase (luc), horseradish peroxidase (HRP) and alkaline phosphatase (AP). Any method known in the art for conjugating a detection moiety to a an antibody may be used.

For indirect IHC assays, the secondary antibody comprising the detectable moiety is applied to the histological sample for 30-120 min at 25° C. or 37° C. to allow formation of immune complexes. In some embodiments, a washing step is used to remove unbound secondary antibody prior to visualization. In some embodiments, a blocking step is used to limit non-specific binding. Any methods for washing or blocking known in the art may be used.

Optimization of conditions for IHC assays is well known in the art. In some embodiments, the antigen binding assay may comprise various techniques for inhibiting non-specific binding. In some embodiments, an antigen binding assay comprises treating the sample with a protein blocking reagent to reduce binding to background proteins that do not comprise the therapeutic target protein. Blocking reagents include, but are not limited to BSA, casein HiBlock (Perkin Elmer, Waltham, Mass.) or protein block (Dako, Carpinteria, Calif.). In some embodiments, the sample is incubated with peroxide to block tissue peroxidases.

Direct or indirect IHC may be used. Direct IHC assays involve using a reagent comprising an indicator moiety, wherein the reagent binds directly to the target. For example, a primary antibody may be labeled with an enzyme or a fluorescent molecule.

In a preferred embodiment, IHC assays are conducted using a commercially available autostainer. The sample may be prepared with, and stained with the targeting complex using a Ventana™ BenchMark ULTRA™ platform, Ventana™ Discovery™, Dako™ Omnis™ Dako™ Autostainer1_ink48™, Leica™ BOND RX™, Leica™ BOND-IN™ or Leica™ BOND MAX™. For example, the Ventana™ BenchMark ULTRA™ platform can be used to stain the sample with the targeting complex, secondary antibody comprising a detectable moiety, and counterstain.

### Predicting Subject Responsiveness

In some embodiments, the subject is determined to be suitable for treatment due to the presence of expression of the target in the sample. Conversely, subjects lacking expression may be considered unsuitable for treatment with the therapeutic agent.

In some embodiments, a patient is determined to be suitable for treatment if at least 5%, 10%, 15%, 20%, 25%, 30%, 35%, 40%, 45%, 50%, 55%, 60%, 65%, 70%, 75%, 80%, 85%, 90%, 95% or more of all cells in the sample express the target of the therapeutic agent.

In some embodiments, results may be interpreted with a light microscope. In some embodiments, samples will be scored on a scale of 0-10, where 0 is negative and 10 is strongly positive. In other embodiments, samples will be scored on a scale of 0-5, where 0 is negative and 5 is strongly positive. The system for scoring can be adapted depending on the therapeutic agent.

### Systems and Kits of the Invention

In some embodiments, the invention comprises systems (e.g., automated systems) or kits comprising components for performing the methods disclosed herein. In some embodiments, targeting complexes are comprised in systems or kits according to the invention. Methods described herein may also utilize such targeting complex systems or kits. Some embodiments described herein are particularly suitable for automation and/or kits, given the minimal amount of reagents and materials required to perform the methods. In certain embodiments, each of the components of a kit may comprise a self-contained unit that is deliverable from a first site to a second site.

In some embodiments, the invention comprises systems or kits for identification of individuals who may benefit from a therapeutic agent. The systems or kits may in certain embodiments comprise a component for incubating the sample with the targeting complex specific for the epitope of interest, wherein the targeting complex comprises two antibodies: antibody A and antibody B. Antibody A further comprises a potential therapeutic monoclonal antibody or fragment thereof. In certain instances the monoclonal antibody may comprises a variable region specific to an epitope of interest and a human constant region. Antibody B further comprises an antibody that recognizes the human constant region of antibody A. In some embodiments of both the systems and the kits of the invention, Antibody B is either a polyclonal or monoclonal antibody raised in mice or rabbits.

In certain embodiments, the systems and/or kits may further comprise a component for preparing samples. Samples may be formalin fixed and paraffin embedded prior to staining. Systems and/or kits may further comprise a component for sectioning FFPE samples and drying samples onto slides.

In some embodiments, the system may comprise an automated method for staining the samples and detecting the presence of specific epitopes. For example, in some embodiments, a commercial autostainer may be used to stain samples with the targeting complex, labelled secondary antibodies, and counterstain.

Additionally, the systems and/or kits may further comprise a component for determining the amount of detection moiety, wherein the amount of detection moiety determines the subjects suitability for receiving treatment with a particular therapeutic agent. For example, in certain embodiments, the system or kit may comprise and/or require a luminometer or other device for measuring a luciferase enzyme activity.

These systems and kits of the invention include various components. As used herein, the term “component” is broadly defined and includes any suitable apparatus or collection of apparatuses suitable for carrying out the recited method. The components need not be integrally connected or situated with respect to each other in any particular way. The invention includes any suitable arrangements of the components with respect to each other. For example, the components need not be in the same room. But in some embodiments, the components are connected to each other in an integral unit. In some embodiments, the same components may perform multiple functions.

### Computer Systems and Computer Readable Media

In certain embodiments, the invention may comprise a system. The system may include at least some of the compositions of the invention. Also, the system may comprise at least some of the components for performing the method. In certain embodiments, the system is formulated as a kit. Thus, in certain embodiments, the invention may comprise a system for identification of a subject who may benefit from treatment with a therapeutic agent comprising: (i) obtaining a sample; (ii) incubating the sample with a targeting complex, as claimed in any one of claims 1 to 13, to form an antigen-antibody complex; (iii) detecting the antigen-antibody complex; and (iv) predicting subject responsiveness to the therapeutic agent. In yet other embodiments, the invention comprises software for use with the methods or systems.

The system, as described in the present technique or any of its components, may be embodied in the form of a computer system. Typical examples of a computer system include a general-purpose computer, a programmed microprocessor, a microcontroller, a peripheral integrated circuit element, and other devices or arrangements of devices that are capable of implementing the steps that constitute the method of the present technique.

A computer system may comprise a computer, an input device, a display unit, and/or the Internet. The computer may further comprise a microprocessor. The microprocessor may be connected to a communication bus. The computer may also include a memory. The memory may include random access memory (RAM) and read only memory (ROM). The computer system may further comprise a storage device. The storage device can be a hard disk drive or a removable storage drive such as a floppy disk drive, optical disk drive, etc. The storage device can also be other similar means for loading computer programs or other instructions into the computer system. The computer system may also include a communication unit. The communication unit allows the computer to connect to other databases and the Internet through an I/O interface. The communication unit allows the transfer to, as well as reception of data from, other databases. The communication unit may include a modem, an Ethernet card, or any similar device which enables the computer system to connect to databases and networks such as LAN, MAN, WAN and the Internet. The computer system thus may facilitate inputs from a user through input device, accessible to the system through I/O interface.

A computing device typically will include an operating system that provides executable program instructions for the general administration and operation of that computing device, and typically will include a computer-readable storage medium (e.g., a hard disk, random access memory, read only memory, etc.) storing instructions that, when executed by a processor of the server, allow the computing device to perform its intended functions. Suitable implementations for the operating system and general functionality of the computing device are known or commercially available, and are readily implemented by persons having ordinary skill in the art, particularly in light of the disclosure herein.

The computer system executes a set of instructions that are stored in one or more storage elements, in order to process input data. The storage elements may also hold data or other information as desired. The storage element may be in the form of an information source or a physical memory element present in the processing machine.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers, or other network devices may be stored locally and/or remotely, as appropriate. Where a system includes computing devices, each such device can include hardware elements that may be electrically coupled via a bus, the elements including, for example, at least one central processing unit (CPU), at least one input device (e.g., a mouse, keyboard, controller, touch screen, or keypad), and at least one output device (e.g., a display device, printer, or speaker). Such a system may also include one or more storage devices, such as disk drives, optical storage devices, and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or Web browser. It should be appreciated that alternate embodiments may have numerous variations from that described above. For example, customized hardware might also be used and/or particular elements might be implemented in hardware, software (including portable software, such as applets), or both. Further, connection to other computing devices such as network input/output devices may be employed.

Non-transient storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, or any other medium which can be used to store the desired information and which can be accessed by the a system device. Based on the disclosure and teachings provided herein, a person of ordinary skill in the art will appreciate other ways and/or methods to implement the various embodiments.

A computer-readable medium may comprise, but is not limited to, an electronic, optical, magnetic, or other storage device capable of providing a processor with computer-readable instructions. Other examples include, but are not limited to, a floppy disk, CD-ROM, DVD, magnetic disk, memory chip, ROM, RAM, SRAM, DRAM, content-addressable memory (“CAM”), DDR, flash memory such as NAND flash or NOR flash, an ASIC, a configured processor, optical storage, magnetic tape or other magnetic storage, or any other medium from which a computer processor can read instructions. In one embodiment, the computing device may comprise a single type of computer-readable medium such as random access memory (RAM). In other embodiments, the computing device may comprise two or more types of computer-readable medium such as random access memory (RAM), a disk drive, and cache. The computing device may be in communication with one or more external computer-readable mediums such as an external hard disk drive or an external DVD or Blu-Ray drive.

As discussed above, the embodiment comprises a processor which is configured to execute computer-executable program instructions and/or to access information stored in memory. The instructions may comprise processor-specific instructions generated by a compiler and/or an interpreter from code written in any suitable computer-programming language including, for example, C, C++, C#, Visual Basic, Java, Python, Perl, JavaScript, and ActionScript (Adobe Systems, Mountain View, Calif.). In an embodiment, the computing device comprises a single processor. In other embodiments, the device comprises two or more processors. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

The computing device comprises a network interface. In some embodiments, the network interface is configured for communicating via wired or wireless communication links. For example, the network interface may allow for communication over networks via Ethernet, IEEE 802.11 (Wi-Fi), 802.16 (Wi-Max), Bluetooth, infrared, etc. As another example, network interface may allow for communication over networks such as CDMA, GSM, UMTS, or other cellular communication networks. In some embodiments, the network interface may allow for point-to-point connections with another device, such as via the Universal Serial Bus (USB), 1394 FireWire, serial or parallel connections, or similar interfaces. Some embodiments of suitable computing devices may comprise two or more network interfaces for communication over one or more networks. In some embodiments, the computing device may include a data store in addition to or in place of a network interface.

Some embodiments of suitable computing devices may comprise or be in communication with a number of external or internal devices such as a mouse, a CD-ROM, DVD, a keyboard, a display, audio speakers, one or more microphones, or any other input or output devices. For example, the computing device may be in communication with various user interface devices and a display. The display may use any suitable technology including, but not limited to, LCD, LED, CRT, and the like.

The set of instructions for execution by the computer system may include various commands that instruct the processing machine to perform specific tasks such as the steps that constitute the method of the present technique. The set of instructions may be in the form of a software program. Further, the software may be in the form of a collection of separate programs, a program module with a larger program or a portion of a program module, as in the present technique. The software may also include modular programming in the form of object-oriented programming. The processing of input data by the processing machine may be in response to user commands, results of previous processing, or a request made by another processing machine.

While the present invention has been disclosed with references to certain embodiments, numerous modifications, alterations and changes to the described embodiments are possible without departing from the scope and spirit of the present invention, as defined in the appended claims. Accordingly, it is intended that the present invention not be limited to the described embodiments, but that it have the full scope defined by the language of the following claims, and equivalents thereof.

### EXAMPLES

### Example 1. Immunohistochemical Assay for the Identification of Subjects Who May Benefit from Treatment with Trastuzumab

Obtain a 3 mm or smaller section of tissue from cancerous tumor tissue from the breast. Fix tissue in 10% neutral buffered formalin (NBF) in a volume 15 to 20 times the volume of the tissue for no less than 4 hours and no more than 8 hours at room temperature (15-25° C.). Next, embed fixed tissue in paraffin. Paraffin fixed and embedded tissues are stable for at least 2 years and should be stored at 15-25° C. Cut 5 μm thick serial sections from each FFPE sample using a micrometer and mount sections on plus slides (e.g., Superfrost Plus slides). Deparaffenize the sample by heating to 60° C. for 1 hour. Perform heat induced antigen retrieval using citrate buffer at pH 6 and cooking samples for 60 minutes in a steamer. Remove slides from steamer and cool for 5 minutes. Wash samples with PBS buffer 3 times for 3 minutes each time. Block endogenous protein using a casein solution for 10 minutes at room temperature. Wash samples with PBS buffer 3 times for 3 minutes each time. Block samples with 5% FCS in PB buffer for 30 min at room temperature. Dilute trastuzumab targeting complex 1:20 to 1:40 in PBS with 5% FCS. The targeting complex is trastuzumab bound to mouse anti-human IgG that recognizes the human constant region of trastuzumab. Incubate samples with the targeting complex at 4° C. overnight. Wash samples with PBS buffer 3 times for 3 minutes each time.

Cover the samples with anti-mouse HRP-labelled secondary antibody and incubate at room temperature for 30 minutes. Wash samples with PBS buffer 3 times for 3 minutes each time. Prepare DAB by adding 2 drops of DAB-chromogen per 1 ml DAB-substrate buffer and mix together. Cover the samples with the prepared DAB chromogen solution and incubate for 10 min. Wash slides with water and counterstain with hemalaun for 2 minutes. Wash slides with water, dehydrate, and mount in a permanent mounting medium (e.g., Pertex). Review slides under a light microscope to evaluate staining and score samples.

### Example 2. Immunohistochemical Assay with Ventana Benchmark XT XT UltraView DAB for the Identification of Subjects Who May Benefit from Treatment with Trastuzumab

Obtain a 3 mm or smaller section of tissue from cancerous tumor tissue from the breast. Fix tissue in 10% neutral buffered formalin (NBF) in a volume 15 to 20 times the volume of the tissue for no less than 4 hours and no more than 8 hours at room temperature (15-25° C.). Next, embed fixed tissue in paraffin. Paraffin fixed and embedded tissues are stable for at least 2 years and should be stored at 15-25° C. Cut 4 μm thick serial sections from each FFPE sample using a micrometer and mount sections on plus slides (e.g., Superfrost Plus slides). Dry samples onto slides at 80° C. for 15 minutes.

Dilute trastuzumab targeting complex 1:20-1:5-in antibody diluent from Ventana. Fill Ventana antibody dispenser with diluted trastuzumab targeting complex. The Ventana staining procedure includes pretreatment with Cell Conditioner 2 (pH 6) for 60 min, followed by incubation with 1:20-1:50 trastuzumab targeting complex at 37° C. for 32 minutes. Following antibody incubation, perform Ventana standard signal amplification, ultraWash, counter-staining with one drop of Hematoxylin for 4 min and one drop of bluing reagent for 4 min.

For chromogenic detection, use UltraView Universal DAB Detection Kit (Ventana). Remove slides from stainer, wash in water with a drop of detergent and mount. Review slides under a light microscope to evaluate staining and score samples.

### Example 3. Immunohistochemical Assay with Ventana Benchmark XT XT UltraView DAB for the Identification of Subjects Who May Benefit from Treatment with Trastuzumab and Docetaxel

Obtain a 3 mm or smaller section of tissue from cancerous tumor tissue from the breast. Fix tissue in 10% neutral buffered formalin (NBF) in a volume 15 to 20 times the volume of the tissue for no less than 4 hours and no more than 8 hours at room temperature (15-25° C.). Next, embed fixed tissue in paraffin. Paraffin fixed and embedded tissues are stable for at least 2 years and should be stored at 15-25° C. Cut 4 μm thick serial sections from each FFPE sample using a micrometer and mount sections on plus slides (e.g., Superfrost Plus slides). Dry samples onto slides at 80° C. for 15 minutes.

Dilute trastuzumab targeting complex 1:20-1:5-in antibody diluent from Ventana. Dilute docetaxel targeting complex 1:20-1:5-in antibody diluent from Ventana. Fill Ventana antibody dispenser with diluted trastuzumab targeting complex and diluted docetaxel targeting complex. The Ventana staining procedure includes pretreatment with Cell Conditioner 2 (pH 6) for 60 min, followed by incubation with 1:20-1:50 trastuzumab targeting complex and 1:20-1:50 docetaxel targeting complex at 37° C. for 32 minutes. Following antibody incubation, perform Ventana standard signal amplification, ultraWash, counter-staining with one drop of Hematoxylin for 4 min and one drop of bluing reagent for 4 min.

For chromogenic detection, use UltraView Universal DAB Detection Kit (Ventana) and use UltraView Universal AP Detection Kit (Ventana). Remove slides from stainer, wash in water with a drop of detergent and mount. Review slides under a light microscope to evaluate staining and score samples.

1. A targeting complex comprising
(i) antibody A, wherein antibody A is a potential therapeutic monoclonal antibody, or fragment thereof, comprising
(a) a variable region specific to an epitope of interest, and
(b) a human constant region; and
(ii) antibody B, wherein antibody B recognizes the human constant region of antibody A.

2. The targeting complex of claim 1, wherein the monoclonal antibody is IgG.

3. The targeting complex of claim 1, wherein the monoclonal antibody fragment is a Fab fragment.

4. (canceled)

5. The targeting complex of claim 1, wherein the monoclonal antibody is fully human.

6. The targeting complex of claim 1, wherein the monoclonal antibody is chimeric.

7. The targeting complex of claim 1, wherein the monoclonal antibody is humanized.

8. (canceled)

9. The targeting complex of claim 1, wherein antibody B is raised in an animal against the human constant region of antibody A.

10-13. (canceled)

14. A method for making a targeting complex comprising:
(i) reacting antibody A, wherein antibody A is a potential therapeutic monoclonal antibody, or fragment thereof, comprising
(a) a variable region specific to an epitope of interest, and
(b) a human constant region,
with antibody B, wherein antibody B recognizes the human constant region of antibody A; and
(ii) purifying the targeting complex.

15. The method of claim 14, wherein the monoclonal antibody is IgG.

16. The method of claim 14, wherein the monoclonal antibody fragment is a Fab fragment.

17. (canceled)

18. The method of claim 14, wherein the monoclonal antibody is fully human.

19. The method of claim 14, wherein the monoclonal antibody is chimeric.

20. The method of claim 14, wherein the monoclonal antibody is humanized.

21. (canceled)

22. The method of claim 14, wherein antibody B is raised in an animal against the human constant region of antibody A.

23. (canceled)

24. The method of claim 14, wherein antibody B is conjugated to a detection moiety.

25-27. (canceled)

28. A method for identifying subjects likely to benefit from treatment with a therapeutic agent comprising:
(i) obtaining a sample;
(ii) incubating the sample with the targeting complex, as claimed in claim 1, to form an antigen-antibody complex;
(iii) detecting the antigen-antibody complex; and
(iv) predicting whether the subject may be responsive to the therapeutic agent.

29. (canceled)

30. The method of claim 28, wherein the targeting complex comprises:
(i) antibody A, wherein antibody A is a potential therapeutic monoclonal antibody, or fragment thereof, comprising
(a) a variable region specific to an epitope of interest, and
(b) a human constant region; and
(ii) antibody B, wherein antibody B recognizes the human constant region of antibody A.

31. (canceled)

32-39. (canceled)

40. The method of claim 28, wherein the sample is incubated with a plurality of varying targeting complexes.

41. The method of claim 40, wherein the plurality of targeting complexes comprises:
i) AbA1, AbA2, AbA3, and so on, wherein each antibody A is a different potential therapeutic monoclonal antibody, or fragment thereof, comprising
(a) a variable region specific to an epitope of interest, and
(b) a human constant region; and
(ii) AbB1, AbB2, AbB2, and so on, wherein each different antibody B recognizes the human constant region of its corresponding antibody A.

42. (canceled)

43. A system for identifying subjects who may benefit from a therapeutic agent comprising a targeting complex of claim 1.
